(19) United States
(12) Patent Application Publication
(10) Pub. No.: US 2013/0085076 A1
(43) Pub. Date: Apr. 4, 2013

(54) BORRELIA BURGDORFERI BACTERIAL ANTIGEN DIAGNOSIC TEST USING POLYMERIC BAIT CONTAINING CAPTURE PARTICLES

(71) Applicants: Temple Douglas, Leesburg, VA (US); Alessandra Luchini, Burke, VA (US); Claudia Fredolini, Collegno (IT); Davide Tamburro, Rome (IT); Lance Liotta, Bethesda, MD (US)

(72) Inventors: Temple Douglas, Leesburg, VA (US); Alessandra Luchini, Burke, VA (US); Claudia Fredolini, Collegno (IT); Davide Tamburro, Rome (IT); Lance Liotta, Bethesda, MD (US)

(21) Appl. No.: 13/513,568

(22) PCT Filed: Dec. 1, 2010
(86) PCT No.: PCT/US10/58517

§ 371 Date: Dec. 5, 2012

Related U.S. Application Data
(60) Provisional application No. 61/265,442, filed on Dec. 1, 2009.

(51) Int. Cl. G01N 33/569

(52) U.S. Cl. CPC G01N 33/56911
USPC 506/9; 435/7.32; 436/501; 435/7.92

(57) ABSTRACT

The invention relates to both a sensitive method for the capture and detection of low-abundance Borrelia burgdorferi (Bb) bacterial antigens allowing for the diagnosis of Lyme Disease using standard immunoassays. Furthermore, this invention allows the antigen to be identified in a sample of urine, serum, or other biological fluids isolated from humans and animals. The invention provides a method to capture, concentrate, separate and specifically quantify the abundance of Bb antigens using immunoassays. The detection of Bb Outer Surface Protein A is presented as an example of the disclosed invention. High sensitivity levels, low cost and easily collected biofluids allow this technology to reach patients in clinics as well as POC applications for the early detection of Lyme disease prior to seroconversion. A kit containing necessary reagents and the method for diagnosis, monitoring or assessing lyme disease using an immunoassay such as an ELISA, western blot or RPPMA is disclosed.

### REFERENCE TO RELATED APPLICATIONS

This application claims priority to an invention which was disclosed in Provisional Application No. 61/157,775, filed Dec. 1, 2009, entitled “Improved Lyme Disease Diagnostic Testing Using Hydrogel Bait Containing Nanoparticles”. The benefit under 35 USC §119(e) of the United States provisional application is hereby claimed, and the aforementioned application is hereby incorporated herein by reference.

### ACKNOWLEDGMENT OF GOVERNMENT SUPPORT

This invention was made with government support under Grant Number DE-FC52-04NA2545 awarded by the United States Department of Energy, and NCI Grant Number 1R21CA137706-01 awarded by the National Institute of Health, The government has certain rights in the invention.

### FIELD OF THE INVENTION

This invention relates to the diagnosis of Lyme Disease by the capture and concentration of low-abundance analytes in biological samples isolated from mammal subjects.

### BACKGROUND OF THE INVENTION

Lyme disease is a bacterial infection caused by the Borrelia burgdorferi (Bb) from Ixodes ticks to humans and animals. Lyme Disease has become an increasingly common illness, which can be treated with antibiotics if identified early enough. Currently Bb infection is difficult to diagnose due to the inaccuracy of the current diagnostic test methods. As a result, Lyme disease is most often diagnosed by clinical symptoms coupled with a series of tests. Current testing methods consist of an enzyme-linked immunosorbent assay (ELISA) performed as a preliminary test to check for antibodies that react with the proteins of the Lyme disease bacteria, Bb. This test can give false negatives if the concentration of antibodies is too low or the antibodies do not react well with the commercial antigens and false positives if the person has been previously treated for Lyme disease or has antibodies for a similar antigen. If this test gives a positive or equivocal reading, a Western blot is performed using commercially produced antigen proteins that are exposed to patient's serum in order to detect antibodies to specific Bb proteins

The method of the present invention includes the use of hydrogel capture particles to concentrate Bb antigens prior to analysis using standard immunoassays, such as ELISA, western blotting and RPPMA techniques, for improved diagnostic capability. The subject invention technology will be able to eliminate false positives from antibody crossover, eliminate false negatives from low concentration proteins or antibodies, provide for a less invasive method of testing Lyme disease by using urine instead of blood serum, and provide for earlier diagnosis of Lyme disease before symptoms become pronounced. In addition, if urine samples must be transported for testing, incubating the hydrogel capture particles with urine immediately after sample collection prevents bacterial proteins from degradation during transit to the testing site location.

One of the major limitations for diagnosing patients by measuring antigens in urine is the ability to accurately measure low-abundance bacterial antigens in urine samples. Concentrating the bacterial proteins using the method disclosed herein, and therefore raising the sensitivity of diagnostic tools, will prevent misdiagnosis if the Lyme disease bacteria are at a dormant stage and the bacteria is present in very low concentration in blood. Testing for bacterial proteins present in the urine of the patient rather than for the antibody response to the infectious agent in the blood, in addition to the concentration of the proteins by the polymeric capture particles provides a much more accurate and reliable test at an earlier stage than the current commercially available tests.

### SUMMARY OF THE INVENTION

The present invention provides a method for detecting Bb antigens associated with Lyme disease in biological fluids isolated from mammal subjects. The method of this invention provides a means to utilize polymeric capture particles to harvest, concentrate and separate the low abundance antigens from biological fluids (i.e. urine, serum, cerebral spinal fluid) isolated from mammal subjects. The concentrated antigens are then detected using by the formation of immune complexes with specific primary monoclonal antibodies and further secondary antibodies conjugated to detection elements further allowing for qualitative and/or quantitative detection of Bb antigens. This invention further allows for the improved ability to detect bacterial antigens at much lower concentrations than current methods. The method of the present invention provide a reliable, rapid, inexpensive and non-invasive means for the detection of such antigens, in a manner that can enable the tailoring and monitoring of an effective therapeutic regimen.

### BRIEF DESCRIPTION OF THE DRAWING

FIG. 1a. Silver stain SDS-PAGE demonstrating the ability of four of nine different types of dye-functionalized polymeric capture particles (listed above each gel) to concentrate Bb proteins in water. Acid Black 48 dye was determined to be the most effective at concentrating the total protein mix. OspA 31 kDa, OspB 34 kDa, IS=initial solution, S=supernatant, and P=polymeric capture particle.

FIG. 1b. Silver stain SDS-PAGE demonstrating the ability of five of nine different types of dye-functionalized polymeric capture particles (listed above each gel) to concentrate B. burgdorferi proteins in water. Acid Black 48 dye was determined to be the most effective at concentrating the total protein mix. OspA 31 kDa, OspB 34 kDa, IS=initial solution, S=supernatant, and P=polymeric capture particle.

FIG. 2. NIPAm-Acid Black 48 polymeric capture particles sequester and concentrate Bb proteins in synthetic urine in a pH dependent manner. SDS-PAGE silver stain of Bb proteins OspA and OspB post incubation with NIPAm-AB48 polymeric capture particles in synthetic urine at three different pH values: 5, 6 and 7 (lanes 3, 5, and 6 respectively) showed optimal binding polymeric capture particles capacity at pH 5 (lane 3). Supernatant was completely depleted. IS=initial solution, S=supernatant, and P=polymeric capture particle content.

FIG. 3a. Western blot analysis demonstrating the presence of Bb proteins captured from synthetic urine. OspA was detected in the protein mixture. NIPAm-AB48 polymeric capture particles completely depleted the supernatant and concentrated B. burgdorferi proteins present in solution. C=control, i.e. Bb protein solution, S=supernatant and P=polymeric capture particles.

FIG. 3b. Western blot analysis demonstrating the presence of Bb proteins captured from synthetic urine. OspB were detected in the protein mixture. NIPAm-AB48 polymeric capture particles completely depleted the supernatant and concentrated B. burgdorferi proteins present in solution. C=control, i.e. Bb protein solution, S=supernatant and P=polymeric capture particles.

FIG. 3c. Western blot analysis demonstrating the presence of Bb proteins captured from synthetic urine. NIPAm/AB48 polymeric capture particles sequestered and concentrated OspA and OspB spiked in 100 mL of synthetic urine at a concentration of 7 μg/mL, 3.5 μg/mL, 0.7 μg/mL. IS=initial solution, S=supernatant, and P=polymeric capture particles.

FIG. 3d. Western blot analysis demonstrating the presence of Bb proteins captured from synthetic urine. NIPAm/AB48 polymeric capture particles sequestered and concentrated OspA and OspB spiked in 100 mL of synthetic urine at a concentration of d.0.35 μg/mL, 0.07 μg/mL. IS=initial solution, S=supernatant, and P=polymeric capture particles.

FIG. 3e. Western blot analysis demonstrating the presence of Bb proteins captured from synthetic urine. NIPAm/AB48 polymeric capture particles sequestered and concentrated OspA and OspB spiked in 100 mL of synthetic urine at a concentration of 0.07 μg/mL, 0.007 μg/mL, and 0.0007 μg/mL. This study indicates that the minimum detectable concentration of B. burgdorferi protein in 100 mL solution after polymeric capture particles concentration was 7 ng/mL, a protein quantity of 700 μg. IS=initial solution, S=supernatant, and P=polymeric capture particles.

FIG. 4. Western blot analysis showing the control mixture containing 7 ng B burgdorferi protein diluted in water, and the eluate from NIPAm-AB48 polymeric capture particles incubated with the proteins. The band pattern from the tick protein mixture was similar to that from the control protein mixture, indicating that the antibodies reacted with Bb protein in the tick. C=commercial control, E=eluate from the polymeric capture particles.

FIG. 5a. Western blot showing the concentration of Bb proteins from 1 mL of human urine. U=plain human urine, IS=initial solution of Bb proteins spiked in human urine, S=supernatant, and P=polymeric capture particles, E=eluate from the polymeric capture particles.

FIG. 5b. Western blot showing the concentration of Bb proteins from 10 mL human urine. U=plain human urine, IS=initial solution of Bb proteins spiked in human urine, S=supernatant, and P=polymeric capture particles.

FIG. 5c. Western blot band intensity graph showing the concentration of Bb proteins from 10 mL human urine. IS=initial solution of Bb proteins spiked in human urine, P=polymeric capture particles. The band intensity for the proteins OspA and OspB was greatly increased by the capture (more than 100 fold).

FIG. 6a. Capture concentration step coupled to reverse phase protein microarray (RPMA) calibrated immunoassay directed against OspA. A calibration curve for OspA with linear region is shown.

FIG. 6b. Capture particle concentration step coupled to reverse phase protein microarray (RPMA) calibrated immunoassay directed against OspA. After polymer capture particle concentration, no OspA was detectable in the supernatant and the yield of the concentration process was higher than 95%.

### DETAILED DESCRIPTION OF THE INVENTION

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

Lyme disease, a bacterial infection caused by the bacteria Borrelia burgdorferi and transmitted by Ixodes sp. ticks to humans, has become an increasingly common illness. In its early stages, this disease is treatable by appropriate antibiotics, but if left untreated, Lyme disease can lead to serious complications including chronic joint inflammation, cognitive defects, heart irregularities, and memory loss. Lyme disease is most often diagnosed after the infection is well established and the patient has raised an antibody titer against the bacteria Bb. Antibodies specific to Bb proteins can take three to four weeks to develop. Early stage Lyme disease, prior to the appearance of a serologic titer, is extremely difficult to diagnose due to the low sensitivity of current diagnostic tests for Bb. Lyme disease tests fall into three categories: serum antibody tests, antigen tests, and PCR for the bacterial nucleic acid. Following sera-conversion in an infected individual, sera antibodies can be detected which recognize a variety of bacterial antigen proteins by Western blot with the following molecular weights (kDa): 18, 22-25, 28, 30, 39, 41, 45, 58, 66 and 93. According to the Centers for Disease Control and Prevention (CDC), a result is considered positive if the patient's sera antibodies react with five or more proteins. Currently both ELISA and western blotting technologies lack adequate sensitivity. Serological testing alone has been found to only be 77% specific when coupled with symptomatic analysis. Ticks found on a patient may be screened for the presence of B. burgdorferi by means of PCR assays for total tick Bb genes (e.g., OspA and Ly1 Chromosomal gene). However, the presence of a positive tick does not mean that the infection is established in the patient.

Ideally, the optimal Lyme disease screening test would utilize a non invasive body fluid such as urine or saliva, and be sensitive enough to reliably detect Stage 1 disease (prior to seroconversion). Immunoassays to detect the bacterial proteins in the patient's urine have been previously proposed but have been judged unreliable due to inadequate sensitivity and specificity. Polymerase chain reaction (PCR) amplification assays targeting shed microbial Bb genes, such as Outer Surface Protein A (OspA), have not proven suitable for direct urine testing. While it is imperative that Lyme disease be diagnosed before stage 2 of the infection, currently there exists no reliable and sensitive testing option.

Polymeric capture particles have been developed to overcome the major roadblocks to discovery and measurement of blood borne biomarker in early detection of cancer. Harvesting polymeric capture particles perform three functions in one step: 1) molecular size sieving, 2) protein concentration, and 3) protection from degradation. Polymeric capture particles are synthesized by the polymerization of N-isopropylacrylamide (NIPAm) and co-monomers (acrylic acid, AAc, and allylamine, AA) with cross links of N,N′-methylenebisacrylamide (BIS). Chemical baits, such as dye molecules (e.g. Acid Black 48, AB48), with high affinity to proteins were covalently incorporated in the capture particles. The bait molecules exhibited a high affinity for the analyte such that the target molecules are completely sequestered from the solution and trapped within the particles. High molecular weight proteins were excluded from the interior of the particles, due to their size sieving properties. The low molecular weight target proteins (smaller than 40 kDa) that were sequestered by the particles are completely protected from degradation. The particle analyte concentration effect is based on the volumetric ratio. The new limit of detection of an assay, following particle-based concentration, can be estimated by a simple mathematical equation, knowing the sensitivity of the assay, the initial volume of biological fluid, and the output volume of the elution:

L min=L/(V/v)=L/C;

where Lmin is the minimum detectable concentration of analyte in the biological fluid, L is the lowest limit of sensitivity of the assay, V is the starting volume of biological fluid tested, v is the output volume of the eluate, and C=V/v is the concentration factor given by the ratio between the initial volume of biological fluid and the volume of the eluate. Assuming that we mix the capture particles with 10 mL of biological fluid and the protein analytes captured in the capture particles can be eluted in the volume v of 50 mL, the concentration factor C is 200. If the sensitivity of the assay L is 50 μg/mL, Lmin, the minimum detectable concentration of analyte, is 0.25 μg/mL with 10 mL of biological fluid as a starting volume. The present invention provides a method of for utilizing capture particles to increase the sensitivity of an antibody based Lyme Disease diagnostic tests directed towards bacterial proteins in urine.

### Example 1

### Polymer Capture Particle Synthesis

Polymeric captured particles were synthesized containing affinity chemical baits. N-isopropylacrylamide (NIPAm), N,N′-methylenebisacrylamide (BIS) polymeric capture particles were synthesized by precipitation polymerization. Capture particles containing acrylic acid (AAc) and allylamine (AA) as copolymers were synthesized as follows. In order to obtain NIPAm-AAc polymeric capture particles, NIPAm (5.2 g) and BIS (0.40 g) were dissolved in MilliQ water (600 mL). The solution was filtered with a nylon filter membrane, pore size 0.45 mm, thoroughly degassed and purged under nitrogen. Acrylic acid (500 mL) was added and the temperature of the system was raised to 80° C. Ammonium persulfate (KPS, 0.276 g) dissolved in water (5 mL) was added. The reaction was allowed to proceed for 6 h at 80° C. The reaction was allowed to cool to room temperature and stirred overnight. Polymeric capture particles were extensively washed by centrifugation in order to remove un-reacted monomer. Dye (disperse orange 3, acid black 48, coomassie brilliant R-250, acid blue 22, pararosaniline base, and rhodamine 123) coupling to NIPAm-AAc capture particles was performed via amidation of carboxylic acid activated with HBTU/HOBt/NMM. Millimoles of acrylic acid in the capture particles were calculated by titration with 0.1 M NaOH (phenolphthalein as indicator). Aliquots of 10 mL of NIPAm-AAc capture particles (0.150 mmol) were lyophilized and resuspended 10 mL of dimethyl formamide (DMF) under nitrogen atmosphere. The suspension was thoroughly purged and degassed with nitrogen to completely exclude water from the system. The capture particles were activated by adding O-benzotriazole-N,N,N′,N′-tetramethyl-uroniumhexafluoro-phosphate (HBTU, 0.171 g, 0.45 mmol), 1-hydroxybenzotriazole (HOBt, 0.060 g, 0.45 mmol), 4-methylmorpholine (NMM, 0.045 g, 0.45 mmol) to the suspension. The contents of the reaction vessel were allowed to react for 5 min at room temperature under nitrogen atmosphere. After 5 min, two equivalents of the dyes listed above (disperse orange 3 0.073 g, acid black 48 0.199 g, coomassie brilliant R-250 0.248 g, acid blue 22 0.221 g, pararosaniline base 0.091 g, and rhodamine 123 0.114 g) dissolved in 10 mL of DMF were added to the suspension and let react for 48 h at room temperature with vigorous stirring under nitrogen atmosphere. Dye-coupled to polymeric capture particles were thoroughly washed in DMF and water. NIPAm/AA capture particles were prepared as follows: NIPAm (17.8 g) and BIS (0.84 g) were dissolved in water (600 mL) and then passed through a 0.2 mm filter nylon membrane. The solution was extensively purged with nitrogen at room temperature with a medium stirring rate before AA (0.52 g) was added. The solution was heated to 75° C. KPS (0.140 g) in water (1.0 mL) was added to the solution to initiate polymerization. The reaction was maintained at 75° C. under nitrogen for 3 h. The reaction was allowed to cool to room temperature and stirred overnight. Polymeric capture particles were extensively washed via centrifugation. To obtain NIPAm/cibacron blue F3 G-A (CB) capture particles, CB (0.76 g) was dissolved in aqueous sodium carbonate (10 mL, 0.1 M). The NIPAm/AA capture particle suspension (10 mL volume) was purged with nitrogen and solid sodium carbonate (0.106 g) was added to the suspension. The CB solution was then added to the NIPAm/AA capture particle suspension, and the reaction was allowed to proceed for 48 h. The resulting NIPAm/CB capture particles were harvested and washed by centrifugation.

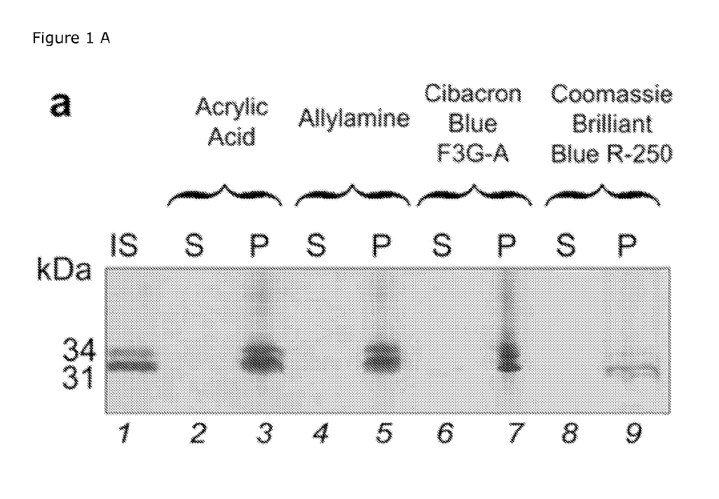

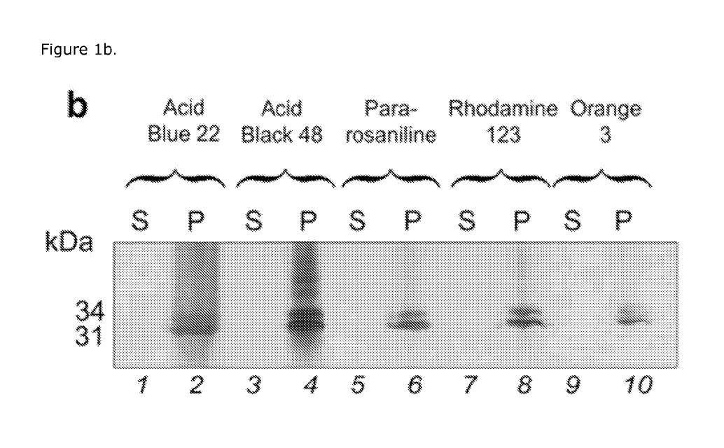

A panel of capture particles functionalized with different affinity baits was screened against B. burgdorferi proteins to determine which capture particles had the best performance. SDS-PAGE analysis was conducted with B. burgdorferi whole protein mixture. Aliquots of 50 ml of Bb protein (0.08 mg/mL) were incubated with 50 ml (1 mg/mL) of nine different types of bait-functionalized capture particles (cibacron blue F3GA, brilliant blue R-250, acid blue 22, disperse orange 3, acid black 48, pararosaniline base, rhodamine 123, acrylic acid, and allylamine). Capture particles were separated from the solution by centrifugation and washed with water prior to loading the SDS-PAGE. Two antigen species at 31 and 34 kDa, corresponding to the known sizes of OspA and OspB, respectively, were sequestered to some degree by every type of bait tested (FIGS. 1a and 1b). Each type of capture particle bait harvested Bb protein with a different level of affinity. AB48 functionalized hydrogel capture particles were chosen for further experimentation because they outperformed all the other classes of baits as demonstrated by the intense silver stained bands on SDS-PAGE.

### Example 2

### Polymeric Capture Particle Characterization

Particle size dependence on temperature and pH was determined via photon correlation spectroscopy (N5 Submicron microparticle Size Analyzer, Beckman Coulter). The pH of the solution was controlled by adding proper amounts of NaOH and HCl. Average values were calculated for three measurements using a 200 s integration time, and the solutions were allowed to thermally equilibrate for 10 min before each set of measurements. Measured values were then converted to capture particle sizes via the Stokese Einstein relationship [26]. Capture particle diameters were measured at increasing temperature from 20° C. to 50° C. in MilliQ water (pH 5.5) and, subsequently, at pH values ranging from 3 to 8 (25° C.). Atomic Force Microscopy (AFM) images of the capture particles were obtained using a NanoInk Atomic Force Microscope (NSCRIPTOR_DPNH System). The NIPAm-AB48 capture particle suspension in MilliQ water (pH 5.5, 1 mg/mL) was sonicated before imaging, was deposited onto a piece of freshly cleaved mica under humid atmosphere at room temperature for 15 min, and was dried under nitrogen flow. Images were acquired under AC mode using a silicon tip with a typical resonance frequency of 300 kHz and a radius smaller than 10 nm. Particle weight was determined by freeze drying the capture particles and weighting the dry particle content.

Particle size and responsiveness to variations in solution temperature and pH were characterized by means of light scattering. At 25° C. NIPAm-acid black 48 (AB48) capture particles had an average diameter of 761.8 nm±16.07. At higher temperatures NIPAm-AB48 microparticle size diminished significantly. The diameter of the capture particles demonstrate a positive correlation with pH. Diameters ranged from 360.8±7.85 nm to 1019.7±33.06 nm for pH values from 2.6 to 8.2. This temperature and pH sensitive behavior is typical of hydrogel capture particles. As a result of the demonstrated dependence of the NIPAm-AB48 capture particle diameter with respect to pH, SDS-PAGE analyses were performed to determine at which pH the capture particles would optimize their performance as described below. All urine samples were then titrated to the optimum pH with hydrochloric acid before incubating the capture particles in the urine. Particles were imaged by means of atomic force microscopy (AFM). NIPAm-AB48 capture particles appear homogeneous in size and not prone to aggregation.

### Example 3

### SDS-PAGE Analysis

Sodium dodecyl sulfate polyacrylamide gel electrophoresis (SDS-PAGE) was performed using 4-20% Tris-Glycine gel in the presence of Tris-Glycine SDS running Buffer on a Novex X-Cell II™ Mini-Cell (Invitrogen Corporation, USA), at 120 V. The gels were stained by silver staining.

Aliquots of capture particles were incubated with analyte solution, synthetic urine (Surine) or human urine, for 30 min at room temperature under slow rotation. Human urine was preliminarily centrifuged at 3000 rcf for 5 min and 4° C. to pellet cellular content. The specific gravity of the urine was measured and noted using a pocket refractometer (PAL-10S Refractometer, Atago Inc.). After incubation, the capture particles were centrifuged (7 min, 25° C., 16,100 rcf), the supernatant was saved and the capture particles were washed three times by resuspending the pellets in water (1 mL) and centrifuging (7 min, 25° C., 16,100 rcf). The capture particles were then directly loaded on the gel or incubated with elution buffers. Incubations with urine (10 mL) were performed with 50 mL polypropylene centrifuge tubes (Nalgene) and capture particles were separated from urine by centrifugation (45 min, 25° C., 40,000 rcf) and washed.

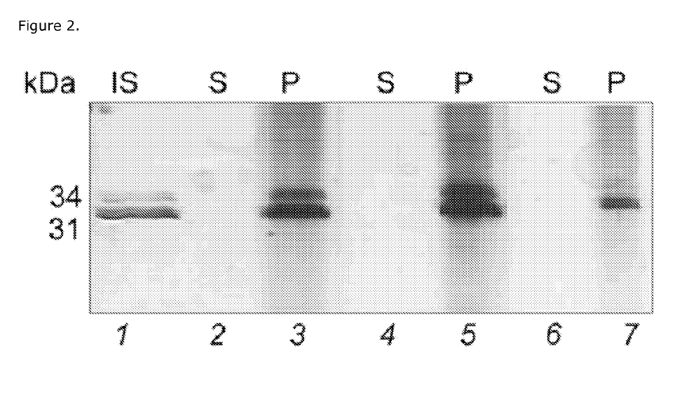

An SDS-PAGE gel electrophoresis experiment was conducted to demonstrate the ability of the NIPAm-AB48 capture particles to concentrate and sequester Bb protein in Surine and to determine the optimum pH of urine for testing. Three solutions were made, each containing 0.088 mg/mL B. burgdorferi whole protein mixture in synthetic urine at pH 5, 6, and 7.50 ml of each solution was mixed with 200 ml of NIPAm-AB48 capture particles. Capture particles were pelleted and washed with water. SDS-PAGE analysis was performed (FIG. 2). NIPAm-Acid Black 48 capture particles were able to concentrate the Bb proteins in synthetic urine and completely deplete the supernatant (the solution phase outside the capture particles). The optimal pH for protein uptake was between 5 and 6. In all subsequent examples, synthetic and human urine were titrated with hydrochloric acid to pH 5 to maintain a constant pH in the range of normal human urine and to optimize the performance of NIPAm-AB48 capture particles.

### Example 4

### Western Blot Analysis

Proteins were separated by 1-D gel electrophoresis in 4-20% Tris-Glycine gel as Example 3, and then transferred onto an Immobilon PVDF membrane. The membrane was then incubated with PBS supplemented with 0.2% I-Block and 0.1% Tween 20 for 1 h at room temperature, and then with antibody raised against OspA and OspB overnight at 4° C. under continuous agitation (1:50 dilution in PBS supplemented with 0.2% I-Block and 0.1% Tween 20). After washes with PBS supplemented with 0.2% I-Block (w/v) and 0.1% Tween 20, immunoreactivity was revealed by using a specific horseradish peroxidase conjugated anti-IgG secondary antibody (1:4000 dilution in PBS supplemented with 0.2% I-Block and 0.1% Tween 20) and the enhanced chemiluminescence system (Supersignal West Dura, ThermoFisher Scientific).

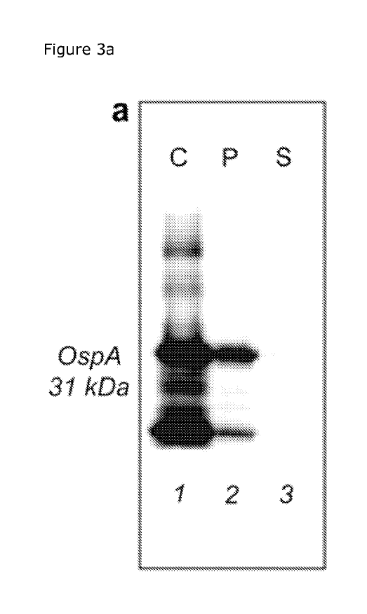

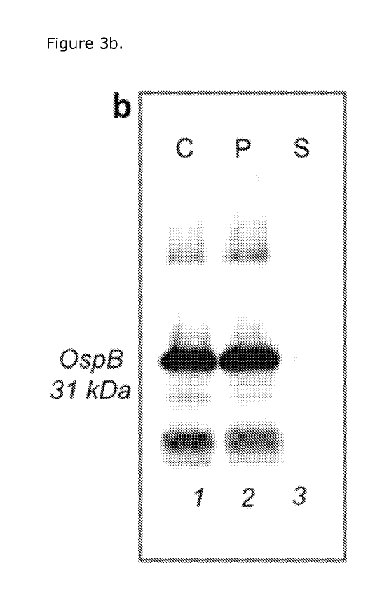

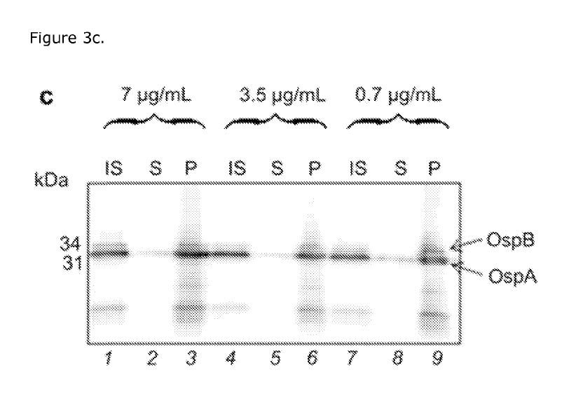

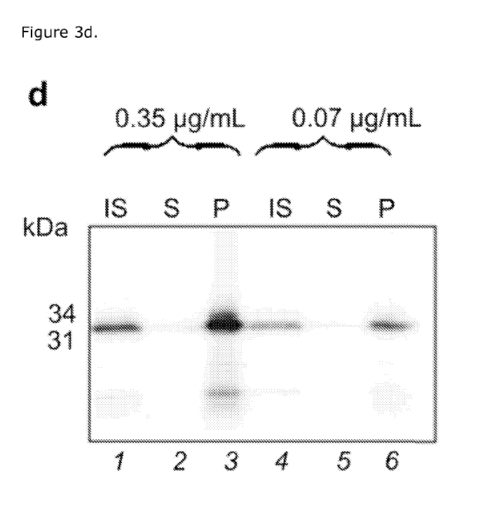

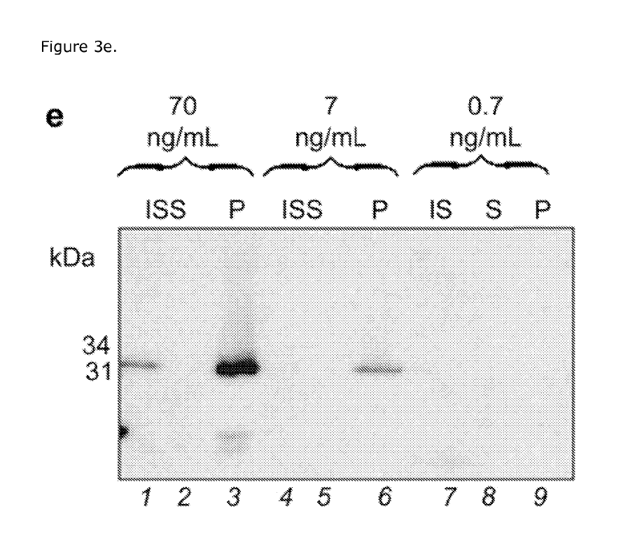

In order to assess the sensitivity of western blotting with NIPAm-AB48 microparticle harvested bacterial proteins, western blotting was performed with Bb proteins dissolved in 100 ml of synthetic urine at pH 5 (0.07 mg/mL) and incubated with 100 ml AB48 capture particles. Capture particles were pelleted by centrifugation, washed, separated by SDS-PAGE and then blotted onto Immobilon PVDF membranes. One membrane was then incubated with OspA antibody and the other with OspB antibody. OspA and OspB antibodies detected strong bands at 31 and 34 kDa respectively (FIG. 3a and b). The analysis confirmed complete uptake of Bb proteins in solution and concentration inside NIPAm-AB48 capture particles.

Additional western blot analyses were performed to test the sensitivity of this system. Bb proteins were dissolved in 100 μL of synthetic urine at pH 5 at the following concentrations 7 μg/mL, 3.5 μg/mL, 0.7 μg/mL, 0.35 μg/mL, 0.070 μg/mL, 0.007 μg/mL, and 0.0007 μg/mL. 100 μL of NIPAm-AB48 capture particles were added to these solutions. NIPAm-AB48 captured Bb proteins with high affinity and concentrated them from synthetic urine (FIGS. 3c-e). The western blot detection limit was 7 ng/mL with the capture particles (FIG. 3e, lane 6) and 70 ng/mL without the capture particles (FIG. 3e, lane 1). The ability of the capture particles to concentrate proteins is based on volumetric ratio between the initial solution and the final capture particle pellet; therefore, the higher the volume of the initial solution, the higher the sensitivity that can be achieved. In this first experiment 100 μL of initial solution was used. If the starting volume is one mL, or ten times larger, the solution will contain ten times more antigen and the sensitivity will increase 10 fold. This effect is due to the fact that capture particles will sequester all of the antigen in this larger volume.

### Example 5

### Protein Extraction from Ixodes Ticks

Ixodes ticks were submerged in liquid nitrogen and pulverized. PBS (1 mL) was added to the pulverized ticks, and the mixture was combined with of NIPAm-AB48 capture particles (100 ml). The washed pellet of capture particles was incubated for 15 min at room temperature with elution buffer (300 ml, 66% acetonitrile-10% ammonium hydroxide). After incubation, the capture particles were spun (7 min, 25° C., 16,100 rcf) and the eluate was saved. The elution step was repeated twice and the eluates were pooled together. Eluates were brought to complete dryness with Speed Vac (Thermo-Fisher).

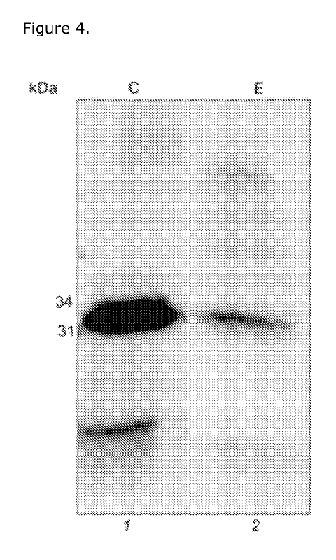

The capture particles were used to detect the Bb antigen in Ixodes ticks. During feeding, Bb reproduces in the tick gut. Bb bacteria require approximately 24 hours to be transferred from tick to human. By the time the Bb bacteria have been transferred to a human, the Bb present in the tick gut have multiplied from approximately 8,000 organisms to approximately 170,000 organisms. Proteins extracted from infected ticks and concentrated by NIPAm-AB48 capture particles contained the characteristic species of Bb and were scored positive for Lyme disease (FIG. 4). To demonstrate the capture particles ability to capture bacterial antigens from tick protein mixtures a total of 14 Ixodes scapularis ticks, representing female and male ticks collected in Virginia and Pennsylvania, were tested for Bb proteins. Each tick was incubated with capture particles in the same procedure as described above, and was tested for the antigens OspA and OspB. Four out of nine female ticks were positive for Lyme disease, while all five male ticks were negative.

### Example 6

### Detection of B. burgdorferi Antigens in Human Urine

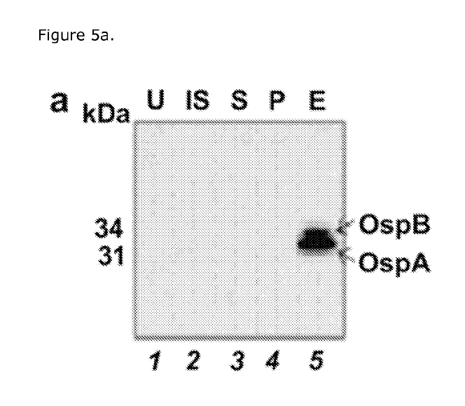

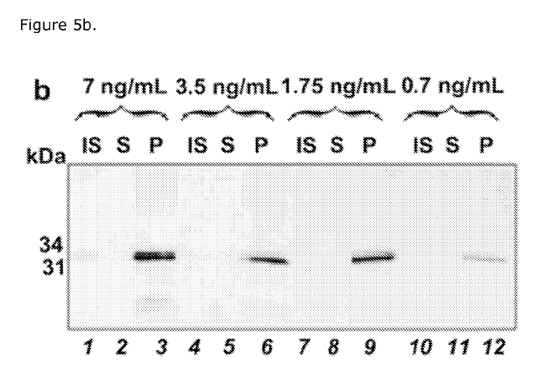

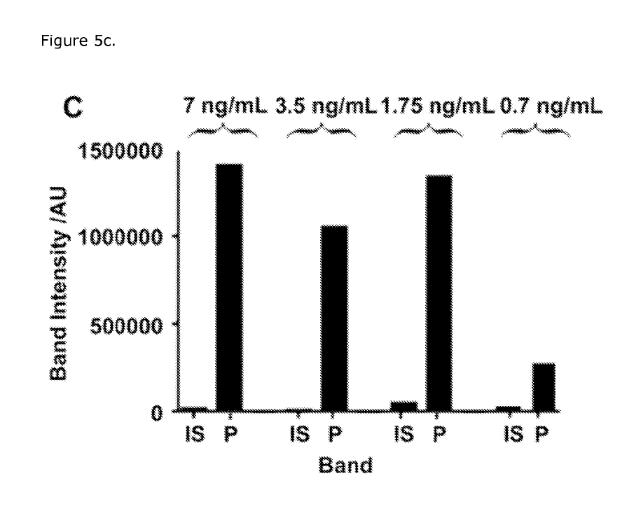

To evaluate the capture particle Lyme Disease detection method in human urine, in the presence of competing human proteins, an incubation was conducted with Bb protein spiked at a concentration of 7 ng/mL in 1 mL of human urine (specific gravity 1.004) titrated at pH 5 using hydrochloric acid. 200 ml of NIPAm-AB48 capture particles were added to the urine solution and incubated for 30 min. Western blot analysis (FIG. 5a) demonstrates that the capture particles were able to completely sequester and concentrate Bb proteins spiked in human urine. Proteins at the initial concentration of 7 ng/mL were not detectable without the capture particle concentration step (FIG. 5a, lane 2); the detection antibodies did not cross react with any of the excess human proteins supporting the specificity of the antibody. To increase the sensitivity, another western blot analysis was performed on a larger volume of urine. Bb proteins were spiked in 10 mL of human urine (specific gravity 1.024) at the following concentrations: 7 ng/mL, 3.5 ng/mL, 1.75 ng/mL, and 0.7 ng/mL. 0.75 mL of NIPAm-AB48 capture particles were added to each sample and the samples were incubated for 30 min (FIG. 5b). The capture particles were able to concentrate 7 ng of Bb proteins from 10 mL to attain an intense western signal in the presence of a 10,000 fold excess of human proteins in the urine. The final concentration of Bb proteins detected in human urine using the capture particles was 0.7 ng/mL. The projected sensitivity limit for western blotting is 700 μg of protein in the entire sample in order for the test to have a positive result. Using ImageQuant 5.2 software, the intensities of the initial solution and capture particle bands on the Western blot shown in FIG. 5b were measured. The capture particles raised the band intensity values of OspA and OspB more than one hundred fold with respect to the initial urine solution achieving the measurement of extremely diluted solutions otherwise not detectable with Western blot analysis (FIG. 5c).

### Example 7

### Reverse Phase Protein Microarray (RPMA) Analysis

Samples were printed on glass backed nitrocellulose array slides using an Aushon 2470 arrayer (Aushon BioSystems, Burlington, Mass.) equipped with 350 mm pins as previously described. Immunostaining was performed on a Dako Autostainer per manufacturer's instructions (CSA kit, Dako). Each slide was incubated with anti-OspA monoclonal antibody (1:50) at room temperature for 30 min. This antibody was validated by western blotting. The negative control slide was incubated with antibody diluent. Secondary antibody was rabbit antimouse IgG (1:10). Subsequent signal detection was amplified via horseradish peroxidase mediated biotinyl tyramide deposition with chromogenic detection (Diaminobenzidine) per manufacturer's instructions (Dako). Total protein per microarray spot was determined with a Sypro Ruby protein stain per manufacturer's directions and imaged with a CCD camera (NovaRay, Alpha Innotech, San Leandro, Calif.).

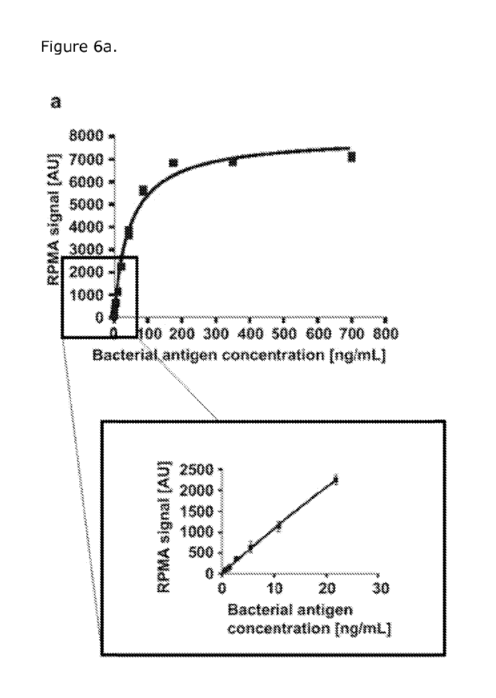

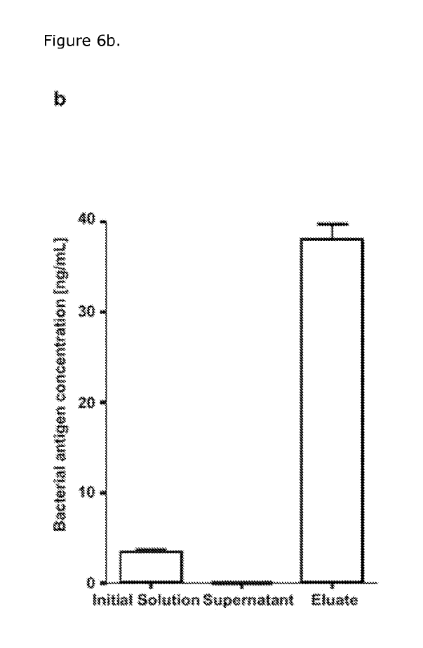

Reverse Phase Protein Microarray (RPMA) technology was used to demonstrate the concentration capabilities of the disclosed method and to provide more sensitive and quantitative information about bacterial antigens in biological solutions. OspA immunoassay using the previously described monoclonal anti-OspA antibody was calibrated as reported in FIG. 6a and the limit of detection was 0.3 ng/mL. Capture particles were incubated with a model solution of OspA diluted in 1 mL of water at a concentration of 3.4±0.4 ng/mL. The captured proteins were eluted in a volume of 100 mL giving a theoretical concentration factor of 10. As shown in FIG. 6b, there was no residual protein in the supernatant and OspA value in the eluate was 38.1±2.3 ng/mL, resulting in a yield higher than 95%.

Using this calibrated immunoassay, we evaluated the presence of bacterial antigen in the urine of an example dog that was infected by Lyme disease. 3.5 mL of dog's urine were incubated with capture particles and the captured protein was reconstituted in a volume of 200 mL (concentration factor of 17.5). RPMA analysis yielded a concentration of OspA in the capture particle processed urine of 1.65±0.2 ng/mL. The estimated concentration of OspA in the original volume of urine from Lyme disease infected dog was 91.4 μg/mL. These data demonstrate disclose method of coupling harvesting capture particles to a quantitative immunoassay for the detection of bacterial proteins in a Lyme disease infected mammal.

What is claimed is:

1. A method of diagnosing, monitoring or assessing the severity of Lyme disease in a mammal, the method comprising the steps of:
a. Obtaining a biological fluid or tissue sample from said mammal,
b. Contacting said sample with multifunctional polymeric molecular capture particles, under conditions in which one or more Borrelia burgdorferi antigens are captured, concentrated and fractionated from other high-abundance interfering molecules or carrier proteins.
c. Contacting said antigens with at least one antibody or antigen binding fragment, under conditions in which immune complexes will form between said antibody or fragment and any antigens associated with Borrelia burgdorferi that may be present in said sample, and
d. Contacting said Borrelia burgdorferi antigen-primary antibody immune complex with a secondary antibody conjugated to a detection label, under certain conditions forming a complex between the Borrelia burgdorferi antigen-primary antibody immune complex and the secondary antibody.
e. Measuring the amount of Borrelia burgdorferi antigens bound to the antibody complex. Detecting the presence of said antigens as a means of diagnosing Lyme disease in said mammals.

2. The method of claim 1, wherein said biological sample is isolated from a human subject.

3. The method of claim 1, wherein said fluid is comprised of one or more of the group consisting of urine, whole blood, serum, plasma, cerebral spinal fluid, sweat, saliva and tissue lysates.

4. The method of claim 1, wherein said mammal is a human.

5. The method of claim 1, wherein said mammal is a canine, equine or feline.

6. The method of claim 1, wherein the polymeric capture particles are comprised of a) a molecular sieve portion; and b) an analyte binding portion; wherein the molecular sieve portion, analyte binding portion or both further comprise a cross-linked region having modified porosity.

7. The method of claim 6, wherein the molecular sieve portion is an outer shell enclosing an inner core, said inner core comprising the analyte binding portion.

8. The method of claim 6, wherein the molecular sieve portion, analyte binding portion or both comprise a hydrogel.

9. The method of claim 6, wherein said analyte binding portion comprises at least one type of moiety capable of chemically or electrostatically binding or sequestering an analyte.

10. The method of claim 6, wherein the analyte binding portion comprises an affinity dye, carboxy group, amine group, thiol group, acrylic acid, antibodies, antibody fragments, synthetic peptides, binding proteins, aptamers, organic compounds, hydrophobic surface, hydrophilic surface, any derivative thereof or any combination thereof.

11. The method of claim 1, wherein said primary antibodies or antibody fragments are immobilized on a solid phase either prior to or following contacting with said test sample.

12. The method of claim 1, wherein said Borrelia burgdorferi antigen is Outer Surface Protein A (OspA)—31 kDa.

13. The method of claim 1, wherein said Borrelia burgdorferi antigen is selected from the group consisting of Outer Surface Protein A (OspA), Outer Surface Protein B (OspB), Outer Surface Protein C (OspC), Outer Surface Protein D (OspD), Outer Surface Protein E (OspE), Outer Surface Protein F (OspF), p35, p39, p41-g, c6, or V1sE.

14. The method of claim 1, wherein said detectable label is selected from the group consisting of a colorimetric label, a radioactive label, an enzymatic label, a chemiluminescent label, a fluorescence label or a thermometric label.

15. The method of claim 1, wherein the detection step (d) further comprises a comparison with a standard curve to determine the quantity of Borrelia burgdorferi antigens present in the sample.

16. The method of claim 11, wherein the solid phase comprises one or more of the following: a substrate, a microwell plate, a bead, a test strip, a later flow device, a protein microarray, a column, nitrocellulose substrate, cellulose substrate, the surface of a tube, a vial, or a sample collection container.

17. The method of claim 1, wherein the antibodies that exhibit specificity for one or more Borrelia burgdorferi antigens are not specific for other tick-borne bacterial antigens.

18. The method of claim 1, wherein the antibodies that exhibit specific for one or more Borrelia burgdorferi antigens are specific for other tick-borne bacterial antigens.

19. The method of claim 1, wherein multiple primary and secondary antibodies are utilized to detect two or more specific antigens.

20. An immunoassay kit for detecting Borrelia burgdorferi antigens in a biological sample, the kit comprising:
(a) Multifunctional polymeric capture particles containing a molecular sieve portion and an inner core comprising the analyte binding portion.
(b) A solid phase substrate for performing the monoclonal antibody-based assay.
(c) A primary monoclonal antibody for detecting Bb antigens.
(d) A secondary anti-monoclonal antibody conjugate,
(e) A detection reagent,
(f) A set of standard assay calibration proteins, and a
(g) Protocol for conducting the assay.

21. The kit of claim 20, wherein the capture particles are comprised of a) a molecular sieve portion; and b) an analyte binding portion; wherein the molecular sieve portion, analyte binding portion or both further comprise a cross-linked region having modified porosity.

22. The kit of claim 21, wherein the molecular sieve portion is an outer shell enclosing an inner core, said inner core comprising the analyte binding portion.

23. The kit of claim of claim 20, wherein the molecular sieve portion, analyte binding portion or both comprise a hydrogel.

24. The kit of claim 20, wherein said analyte binding portion comprises at least one type of moiety capable of chemically or electrostatically binding or sequestering an analyte.

25. The kit of claim 20, wherein the analyte binding portion comprises an affinity dye, carboxy group, amine group, thiol group, acrylic acid, antibodies, antibody fragments, synthetic peptides, binding proteins, aptamers, organic compounds, hydrophobic surface, hydrophilic surface, any derivative thereof or any combination thereof.

26. The kit of claim 20, wherein the solid phase substrate comprises one or more of the following: a substrate, a microwell plate, a bead, a test strip, a later flow device, a protein microarray, a column, nitrocellulose substrate, cellulose substrate, the surface of a tube, a vial, or a sample collection container.

27. The kit of claim 20, further comprising purified Borrelia Burgdorferi antigens as antigen calibration standards.

28. The kit of claim 20, wherein the antigens in step c are comprised of one or more of the group consisting of OspA, OspB, OspC, OspD, OspE, OspF, p35, p39, p41-g, c6 and V1sE.

29. The kit of claim 21, wherein said detection reagent is a detectable label is selected from the group consisting of a colorimetric label, a radioactive label, an enzymatic label, a chemiluminescent label, a fluorescence label or a thermometric label.

30. A method of diagnosing, monitoring or assessing the severity of tick-borne disease in a mammal, the method comprising the steps of:
a. Obtaining a biological fluid or tissue sample from said mammal,
b. Contacting said sample with multifunctional polymeric molecular capture particles, under conditions in which one or more bacterial antigens are captured, concentrated and fractionated from other high-abundance interfering molecules or carrier proteins.
c. Contacting said antigens with at least one primary antibody or antigen binding fragment, under conditions in which specific immune complexes will form between said antibody or fragment and any antigens associated with the specific bacterial infection that may be present in said sample, and
d. Contacting said antigen-primary antibody immune complex with a secondary antibody conjugated to a detection label, under certain conditions forming a complex between the antigen-primary antibody immune complex and the secondary antibody.
e. Detecting the presence of said antigens as a means of diagnosing tick-borne bacterial infections in said mammals.

31. The method of claim 30, wherein said biological sample is isolated from a mammal subject or an arthropod.

32. The method of claim 30, where said tick-borne disease may include one or more of the group consisting of Anaplasmosis, Babesiosis, Ehrilichiosis, Richettsiosis, Rocky Mountain Spotted Fever (RMSF), Southern Tick-Associated Rash Illness, Tularemia, and Crimean-Congo hemorrhagic fever.

33. An immunoassay kit for detecting tick-bone disease antigens in a biological sample, the kit comprising:
(h) As multifunctional molecular capture particles, core-shell polymeric particles containing a molecular sieve portion and an inner core comprising the analyte binding portion.
(i) A solid support substrate for performing the monoclonal antibody-based assay.
(j) A primary monoclonal antibody for detecting antigens.
(k) A secondary anti-monoclonal antibody conjugate,
(l) A detection reagent,
(m) A set of standard assay calibration proteins, and a
(n) Protocol for conducting the assay.
